SEQUENCE LISTING (192 sequences)

SEQ ID NO: 1 - DNA, 40 bp, Artificial Sequence
  Description of Artificial Sequence SNP3 foward primer.
aggtgtcagacataccctctttttggagatttcctgttcc

SEQ ID NO: 2 - DNA, 36 bp, Artificial Sequence
  Description of Artificial Sequence SNP3 reverse primer.
aattgctctgctcttgtaagtctgggatgctttcct

SEQ ID NO: 3 - DNA, 42 bp, Artificial Sequence
  Description of Artificial Sequence SNP4 foward primer.
agtacttgatcctgtatttcaccatcatcccatcacctacct

SEQ ID NO: 4 - DNA, 45 bp, Artificial Sequence
  Description of Artificial Sequence SNP4 reverse primer.
ggccctttttgtaaatggaggatctctggtgagtcctagtaaatt

SEQ ID NO: 5 - DNA, 40 bp, Artificial Sequence
  Description of Artificial Sequence SNP5 foward primer.
tccctacgtggttctccctcatctaagaagccataaggat

SEQ ID NO: 6 - DNA, 44 bp, Artificial Sequence
  Description of Artificial Sequence SNP5 reverse primer.
atgctaacctttgttttaagccacattccctcttactatgtcct

SEQ ID NO: 7 - DNA, 35 bp, Artificial Sequence
  Description of Artificial Sequence SNP6 foward primer.
tgacttctggtgctagatcatgtcctcattccccg

SEQ ID NO: 8 - DNA, 39 bp, Artificial Sequence
  Description of Artificial Sequence SNP6 reverse primer.
actctttctccatggcatctacttttcgaggacctagct

SEQ ID NO: 9 - DNA, 35 bp, Artificial Sequence
  Description of Artificial Sequence SNP7 foward primer.
cagctcctactcagccattcctgaacagaggacag

SEQ ID NO: 10 - DNA, 36 bp, Artificial Sequence
  Description of Artificial Sequence SNP7 reverse primer.
gggatctggtgtattaaatgccctgccttctgatgg

SEQ ID NO: 11 - DNA, 50 bp, Artificial Sequence
  Description of Artificial Sequence SNP8 foward primer.
tggagcgatttcctatttaccaaagagagctaaagttcataattctacag

SEQ ID NO: 12 - DNA, 47 bp, Artificial Sequence
  Description of Artificial Sequence SNP8 reverse primer.
ctgcattaaatggaaaaactttaaaagaatgcatcctaagggcagct

SEQ ID NO: 13 - DNA, 41 bp, Artificial Sequence
  Description of Artificial Sequence SNP9 foward primer.
aataaatggtagagctgagattcaaactgaagcagtggcct

SEQ ID NO: 14 - DNA, 48 bp, Artificial Sequence
  Description of Artificial Sequence SNP9 reverse primer.
ccctaaaagttgcaaaccataatttcatttgtcaagtaaacatagcca

SEQ ID NO: 15 - DNA, 45 bp, Artificial Sequence
  Description of Artificial Sequence SNP10 foward primer.
aggcatgcttcactttaagaaaatctaacacaaaaacctgaacca

SEQ ID NO: 16 - DNA, 42 bp, Artificial Sequence
  Description of Artificial Sequence SNP10 reverse primer.
gcgtacctagcacataatttttttcttgtaatgtgggcgtgt

SEQ ID NO: 17 - DNA, 36 bp, Artificial Sequence
  Description of Artificial Sequence SNP11 foward primer.
cagcaagatggacaccgtgaaagggcagtcagtttg

SEQ ID NO: 18 - DNA, 38 bp, Artificial Sequence
  Description of Artificial Sequence SNP11 reverse primer.
ccgtcattctcgggatccatttcctcctgggatatttg

SEQ ID NO: 19 - DNA, 36 bp, Artificial Sequence
  Description of Artificial Sequence SNP12 foward primer.
tgccagccttctcagaaatctctcgtcgacctactc

SEQ ID NO: 20 - DNA, 44 bp, Artificial Sequence
  Description of Artificial Sequence SNP12 reverse primer.
accaccacatatcttaaagctgacaacctaacatctttcaacat

SEQ ID NO: 21 - DNA, 35 bp, Artificial Sequence
  Description of Artificial Sequence SNP13 foward primer.
tcggatctcctacttctctctccctccctccctct

SEQ ID NO: 22 - DNA, 35 bp, Artificial Sequence
  Description of Artificial Sequence SNP13 reverse primer.
gatgagcaaggtcacactacgtcagagatgggccc

SEQ ID NO: 23 - DNA, 38 bp, Artificial Sequence
  Description of Artificial Sequence SNP14 foward primer.
taagttttccatcaggcagcagttgagcaggaacgcaa

SEQ ID NO: 24 - DNA, 36 bp, Artificial Sequence
  Description of Artificial Sequence SNP14 reverse primer.
ggaatcagtaagtcctactgctgttcaggccctgac

SEQ ID NO: 25 - DNA, 35 bp, Artificial Sequence
  Description of Artificial Sequence SNP15 foward primer.
gcagtcacctctaagctggataccacttctgccag

SEQ ID NO: 26 - DNA, 47 bp, Artificial Sequence
  Description of Artificial Sequence SNP15 reverse primer.
ctgagtatggacttggatgatcaacttggttaatattcaggctatct

SEQ ID NO: 27 - DNA, 36 bp, Artificial Sequence
  Description of Artificial Sequence SNP16 foward primer.
gtttgtttatcagctgcctcaccccaaactgtttgc

SEQ ID NO: 28 - DNA, 35 bp, Artificial Sequence
  Description of Artificial Sequence SNP16 reverse primer.
cagtgagtgtgagtgggaagatagggatgggagtg

SEQ ID NO: 29 - DNA, 40 bp, Artificial Sequence
  Description of Artificial Sequence SNP17 foward primer.
acctcctatcccctattaccttgtcacctggatttttgtt

SEQ ID NO: 30 - DNA, 39 bp, Artificial Sequence
  Description of Artificial Sequence SNP17 reverse primer.
gtctttttggaaagctgtgaggggcctggtattaggaca

SEQ ID NO: 31 - DNA, 35 bp, Artificial Sequence
  Description of Artificial Sequence SNP18 foward primer.
tgtcctaataccaggcccctcacagctttccaaaa

SEQ ID NO: 32 - DNA, 37 bp, Artificial Sequence
  Description of Artificial Sequence SNP18 reverse primer.
aatttgcaaggtccagggcaaactgaaaatgcagaac

SEQ ID NO: 33 - DNA, 43 bp, Artificial Sequence
  Description of Artificial Sequence SNP19 foward primer.
ggccatacagttttagaggattcatttccctttctcctgaagc

SEQ ID NO: 34 - DNA, 43 bp, Artificial Sequence
  Description of Artificial Sequence SNP19 reverse primer.
agctaagggaattggggaaaatggaaaataaagaggagggaag

SEQ ID NO: 35 - DNA, 35 bp, Artificial Sequence
  Description of Artificial Sequence SNP20 foward primer.
cccttcacctggacacacttttgttccattctccc

SEQ ID NO: 36 - DNA, 36 bp, Artificial Sequence
  Description of Artificial Sequence SNP20 reverse primer.
tttttaaccttggctgcacttggaatcacccggaga

SEQ ID NO: 37 - DNA, 38 bp, Artificial Sequence
  Description of Artificial Sequence SNP21 foward primer.
gtattttttccaagctctccgggtgattccaagtgcag

SEQ ID NO: 38 - DNA, 44 bp, Artificial Sequence
  Description of Artificial Sequence SNP21 reverse primer.
cctgccaaacctagagaatgcagtataacacaaaacatgagatg

SEQ ID NO: 39 - DNA, 38 bp, Artificial Sequence
  Description of Artificial Sequence SNP22 foward primer.
tctgagttgtgtaggcttgagacttcttcctggttgct

SEQ ID NO: 40 - DNA, 39 bp, Artificial Sequence
  Description of Artificial Sequence SNP22 reverse primer.
cactgtgacaatagggacaaattgggatggtactgagcc

SEQ ID NO: 41 - DNA, 38 bp, Artificial Sequence
  Description of Artificial Sequence SNP23 foward primer.
cttcttgcctcaaaagtccgaatctggttaggtgactc

SEQ ID NO: 42 - DNA, 37 bp, Artificial Sequence
  Description of Artificial Sequence SNP23 reverse primer.
ggcttagggaggatcgtcacatggaagatggattctg

SEQ ID NO: 43 - DNA, 35 bp, Artificial Sequence
  Description of Artificial Sequence SNP24 foward primer.
gggcacagggagcttcaggccagtgttgttggaaa

SEQ ID NO: 44 - DNA, 36 bp, Artificial Sequence
  Description of Artificial Sequence SNP24 reverse primer.
ggtcttacctactgactggctggcttgactgatctt

SEQ ID NO: 45 - DNA, 42 bp, Artificial Sequence
  Description of Artificial Sequence SNP25 foward primer.
tggtagttttactccattgtgctctgcaggaaattactggct

SEQ ID NO: 46 - DNA, 40 bp, Artificial Sequence
  Description of Artificial Sequence SNP25 reverse primer.
agcagcacaaaacaaggactcatttctgcactactttgac

SEQ ID NO: 47 - DNA, 38 bp, Artificial Sequence
  Description of Artificial Sequence SNP26 foward primer.
gaggaaatgaccactcgggaccaaggacactaacccta

SEQ ID NO: 48 - DNA, 39 bp, Artificial Sequence
  Description of Artificial Sequence SNP26 reverse primer.
aggaagatgccatcacttacacacctatcaaaaggagga

SEQ ID NO: 49 - DNA, 41 bp, Artificial Sequence
  Description of Artificial Sequence SNP27 foward primer.
agtactggctgtttctatcagtccaggtttttaaatggcct

SEQ ID NO: 50 - DNA, 35 bp, Artificial Sequence
  Description of Artificial Sequence SNP27 reverse primer.
cgggcactgctttttgggaaaggacatgcaggcga

SEQ ID NO: 51 - DNA, 49 bp, Artificial Sequence
  Description of Artificial Sequence SNP28 foward primer.
ggctccattaaaatctcatagaaattcaaccttctataagttgccaagt

SEQ ID NO: 52 - DNA, 41 bp, Artificial Sequence
  Description of Artificial Sequence SNP28 reverse primer.
actgacttgccaagaatttctgttcctgttcaaaacaaggg

SEQ ID NO: 53 - DNA, 35 bp, Artificial Sequence
  Description of Artificial Sequence SNP29 foward primer.
gagagtttttgcctcattgtgggtgggcctgtgtg

SEQ ID NO: 54 - DNA, 40 bp, Artificial Sequence
  Description of Artificial Sequence SNP29 reverse primer.
agcaaaggaacaggaaatctccaaaaagagggtatgtctg

SEQ ID NO: 55 - DNA, 35 bp, Artificial Sequence
  Description of Artificial Sequence SNP30 foward primer.
gaggaacgaacagctctgatccattagccctccag

SEQ ID NO: 56 - DNA, 48 bp, Artificial Sequence
  Description of Artificial Sequence SNP30 reverse primer.
agggccactggtatctctagttttgaattgctttgatttttttttggt

SEQ ID NO: 57 - DNA, 40 bp, Artificial Sequence
  Description of Artificial Sequence SNP31 foward primer.
acagattctcaggcctctgtttaaaagagtcgaagtaggc

SEQ ID NO: 58 - DNA, 39 bp, Artificial Sequence
  Description of Artificial Sequence SNP31 reverse primer.
tgccttttctctcttgcactatttggattgtagagcccc

SEQ ID NO: 59 - DNA, 42 bp, Artificial Sequence
  Description of Artificial Sequence SNP32 foward primer.
acaaacaaataaacagagaagtgtaaaaagcagacgtggcct

SEQ ID NO: 60 - DNA, 46 bp, Artificial Sequence
  Description of Artificial Sequence SNP32 reverse primer.
ccaggtcacttattatttaccatagcagcaaagacataatggtcat

SEQ ID NO: 61 - DNA, 43 bp, Artificial Sequence
  Description of Artificial Sequence SNP33 foward primer.
aggaaaaaagaaaattcctactctatgatggcagcacacacac

SEQ ID NO: 62 - DNA, 43 bp, Artificial Sequence
  Description of Artificial Sequence SNP33 reverse primer.
aggctgaaatgtaagaactaagaagcccatgtacctaggacac

SEQ ID NO: 63 - DNA, 36 bp, Artificial Sequence
  Description of Artificial Sequence SNP34 foward primer.
agagcgattcacaccatccctttgtcatttttacct

SEQ ID NO: 64 - DNA, 37 bp, Artificial Sequence
  Description of Artificial Sequence SNP34 reverse primer.
caggtaccagttgaatgaagagaagcacacctcctcc

SEQ ID NO: 65 - DNA, 35 bp, Artificial Sequence
  Description of Artificial Sequence SNP35 foward primer.
atccattggtctggtcatgctgggaaaatttggga

SEQ ID NO: 66 - DNA, 42 bp, Artificial Sequence
  Description of Artificial Sequence SNP35 reverse primer.
cctgtgtacatgatgttgtgcaaaacaaaatgtctgtagcgc

SEQ ID NO: 67 - DNA, 36 bp, Artificial Sequence
  Description of Artificial Sequence SNP36 foward primer.
agaaatgcagacacacctcatcaattgggcaattgg

SEQ ID NO: 68 - DNA, 36 bp, Artificial Sequence
  Description of Artificial Sequence SNP36 reverse primer.
cagaggaacagcacacccatggatttcatttgaccc

SEQ ID NO: 69 - DNA, 39 bp, Artificial Sequence
  Description of Artificial Sequence SNP37 foward primer.
acctatcatagccaaaacctatccctgaaacatcctggt

SEQ ID NO: 70 - DNA, 41 bp, Artificial Sequence
  Description of Artificial Sequence SNP37 reverse primer.
agtcctacttaccatttcactttctgcatctgctctaaggt

SEQ ID NO: 71 - DNA, 40 bp, Artificial Sequence
  Description of Artificial Sequence SNP38 foward primer.
agctaaaagttccaaccctctaatcctcaaatgacctggt

SEQ ID NO: 72 - DNA, 40 bp, Artificial Sequence
  Description of Artificial Sequence SNP38 reverse primer.
gggctaaagctgttgacctgatagatatgggttcaagagg

SEQ ID NO: 73 - DNA, 41 bp, Artificial Sequence
  Description of Artificial Sequence SNP39 foward primer.
gaacaaaatggcttgttacagacgaaaatgtggaaagggca

SEQ ID NO: 74 - DNA, 35 bp, Artificial Sequence
  Description of Artificial Sequence SNP39 reverse primer.
ggcagcaggcgacccaagaccgtccgcggagggta

SEQ ID NO: 75 - DNA, 35 bp, Artificial Sequence
  Description of Artificial Sequence SNP40 foward primer.
cttgggaacgcttcacgaggtgacctccagccaca

SEQ ID NO: 76 - DNA, 40 bp, Artificial Sequence
  Description of Artificial Sequence SNP40 reverse primer.
actagaggaaaaagggaagaggcagttgtatacacatgct

SEQ ID NO: 77 - DNA, 43 bp, Artificial Sequence
  Description of Artificial Sequence SNP41 foward primer.
agcagttgttggagctggatgtaggaacatgatgttaatgatg

SEQ ID NO: 78 - DNA, 39 bp, Artificial Sequence
  Description of Artificial Sequence SNP41 reverse primer.
ctctaacctgcgaattggtcatctgcacatttagtgagc

SEQ ID NO: 79 - DNA, 42 bp, Artificial Sequence
  Description of Artificial Sequence SNP42 foward primer.
agatccaagtgtgtgtgcatgtgtgtgttttttctttttgga

SEQ ID NO: 80 - DNA, 42 bp, Artificial Sequence
  Description of Artificial Sequence SNP42 reverse primer.
agactgacaacatataagtaggtgaaagggcacataactcct

SEQ ID NO: 81 - DNA, 50 bp, Artificial Sequence
  Description of Artificial Sequence SNP43 foward primer.
gcaggataaaatgtgtgtaggaatataggaatacatggcaatcagtaaca

SEQ ID NO: 82 - DNA, 46 bp, Artificial Sequence
  Description of Artificial Sequence SNP43 reverse primer.
acctcatacataattggcactgctttttcagttatgagaagtagag

SEQ ID NO: 83 - DNA, 41 bp, Artificial Sequence
  Description of Artificial Sequence SNP44 foward primer.
tctatgtttgcccttgtgttcagttttgagacctagcacct

SEQ ID NO: 84 - DNA, 38 bp, Artificial Sequence
  Description of Artificial Sequence SNP44 reverse primer.
caaagggtcagcagcggtttctcacaggactatcatga

SEQ ID NO: 85 - DNA, 35 bp, Artificial Sequence
  Description of Artificial Sequence SNP45 foward primer.
tgagcctagtatagttggtgtgcagcatcagggat

SEQ ID NO: 86 - DNA, 37 bp, Artificial Sequence
  Description of Artificial Sequence SNP45 reverse primer.
cattgtaggccccactgtcttgcttccattttgagga

SEQ ID NO: 87 - DNA, 39 bp, Artificial Sequence
  Description of Artificial Sequence SNP46 foward primer.
agaaccttcttgccatgtgacacaatgattacctgagga

SEQ ID NO: 88 - DNA, 47 bp, Artificial Sequence
  Description of Artificial Sequence SNP46 reverse primer.
ggctttgacaacccaggagtactttaattgctcttgaatttcagaca

SEQ ID NO: 89 - DNA, 39 bp, Artificial Sequence
  Description of Artificial Sequence SNP47 foward primer.
ccgcactatactgtgaatatcattgagagcttggtccct

SEQ ID NO: 90 - DNA, 36 bp, Artificial Sequence
  Description of Artificial Sequence SNP47 reverse primer.
gagagaggatgctgtaattgggatggggcacatgga

SEQ ID NO: 91 - DNA, 48 bp, Artificial Sequence
  Description of Artificial Sequence SNP48 foward primer.
acagggacagaaattcttggcaagtcagttcgtataatattctctacg

SEQ ID NO: 92 - DNA, 44 bp, Artificial Sequence
  Description of Artificial Sequence SNP48 reverse primer.
gggattctgttttgttttggctcttttgagtgtggagaaaacat

SEQ ID NO: 93 - DNA, 35 bp, Artificial Sequence
  Description of Artificial Sequence SNP49 foward primer.
gttagaagctgtaaatgctgcctggtggggttctg

SEQ ID NO: 94 - DNA, 37 bp, Artificial Sequence
  Description of Artificial Sequence SNP49 reverse primer.
ccaaaaccacctaacttaaaagccctctgccacactc

SEQ ID NO: 95 - DNA, 39 bp, Artificial Sequence
  Description of Artificial Sequence SNP50 foward primer.
tgttcctgcagttacaagacctaagttccaagaagcagc

SEQ ID NO: 96 - DNA, 35 bp, Artificial Sequence
  Description of Artificial Sequence SNP50 reverse primer.
gagcaaaagctagcgatgtgcacttggacatgttt

SEQ ID NO: 97 - DNA, 39 bp, Artificial Sequence
  Description of Artificial Sequence SNP51 foward primer.
ggtctggtttgtcaaagagtgtgggtcatcaacagagag

SEQ ID NO: 98 - DNA, 43 bp, Artificial Sequence
  Description of Artificial Sequence SNP51 reverse primer.
catagatcaaaagataattttccccagcccaagtggtacagtg

SEQ ID NO: 99 - DNA, 43 bp, Artificial Sequence
  Description of Artificial Sequence SNP52 foward primer.
aggtagccatttccaataatttatagaacagttcatgggccct

SEQ ID NO: 100 - DNA, 44 bp, Artificial Sequence
  Description of Artificial Sequence SNP52 reverse primer.
agccaaataatacaagtctggagaagcaaaggaaagagggtagg

SEQ ID NO: 101 - DNA, 42 bp, Artificial Sequence
  Description of Artificial Sequence SNP53 foward primer.
atcagagtttaaaaggaacatgaggggaaagatgtccatgca

SEQ ID NO: 102 - DNA, 36 bp, Artificial Sequence
  Description of Artificial Sequence SNP53 reverse primer.
agaaacaaggtgaggatggctgtcaggatggtcaga

SEQ ID NO: 103 - DNA, 41 bp, Artificial Sequence
  Description of Artificial Sequence SNP54 foward primer.
accattgaccagatgctaagagtcaaagggtaagaagacct

SEQ ID NO: 104 - DNA, 45 bp, Artificial Sequence
  Description of Artificial Sequence SNP54 reverse primer.
acacattttaaaaagatgcctcccagtctccaaacaaacaagaac

SEQ ID NO: 105 - DNA, 48 bp, Artificial Sequence
  Description of Artificial Sequence SNP55 foward primer.
tgccaaaaacagtgtcattgtgtgtgttcctttcttgatacttagtca

SEQ ID NO: 106 - DNA, 36 bp, Artificial Sequence
  Description of Artificial Sequence SNP55 reverse primer.
cttccctgttctcctcctgtcctttattgctgctcc

SEQ ID NO: 107 - DNA, 41 bp, Artificial Sequence
  Description of Artificial Sequence SNP56 foward primer.
cacatgtgttcaattttgaggccatttgttgatttttgcgg

SEQ ID NO: 108 - DNA, 37 bp, Artificial Sequence
  Description of Artificial Sequence SNP56 reverse primer.
gtgaaattaataggggtgagaagaggggagggtcagc

SEQ ID NO: 109 - DNA, 39 bp, Artificial Sequence
  Description of Artificial Sequence SNP57 foward primer.
agatgctaagggacaactgtaataggcttctgaatgggg

SEQ ID NO: 110 - DNA, 36 bp, Artificial Sequence
  Description of Artificial Sequence SNP57 reverse primer.
tgtaagtggcaccagggcagggacttgggttgtttt

SEQ ID NO: 111 - DNA, 35 bp, Artificial Sequence
  Description of Artificial Sequence SNP58 foward primer.
tcaaaccagggcaaaggtacattggaagacccaac

SEQ ID NO: 112 - DNA, 35 bp, Artificial Sequence
  Description of Artificial Sequence SNP58 reverse primer.
ccctttggtgattctgatgcaggctgtggacctca

SEQ ID NO: 113 - DNA, 35 bp, Artificial Sequence
  Description of Artificial Sequence SNP59 foward primer.
ctcagaggaaaaagccagacactgactcctgtcca

SEQ ID NO: 114 - DNA, 36 bp, Artificial Sequence
  Description of Artificial Sequence SNP59 reverse primer.
gatgagggagaaccacgtagggatggagaaagcttg

SEQ ID NO: 115 - DNA, 35 bp, Artificial Sequence
  Description of Artificial Sequence SNP60 foward primer.
gcaaggtacccctgacctcttatgctaccagagag

SEQ ID NO: 116 - DNA, 36 bp, Artificial Sequence
  Description of Artificial Sequence SNP60 reverse primer.
gctggtggcagacttgtgtttctggagaagagagtc

SEQ ID NO: 117 - DNA, 35 bp, Artificial Sequence
  Description of Artificial Sequence SNP61 foward primer.
gatggtgaacactgtgtaaccctggcattgtcact

SEQ ID NO: 118 - DNA, 36 bp, Artificial Sequence
  Description of Artificial Sequence SNP61 reverse primer.
gcactctgcagggctccaatcgaacaaatagaagac

SEQ ID NO: 119 - DNA, 46 bp, Artificial Sequence
  Description of Artificial Sequence SNP62 foward primer.
atgcaggaaaagagacaaaatgcacagataccctaatttcagaact

SEQ ID NO: 120 - DNA, 35 bp, Artificial Sequence
  Description of Artificial Sequence SNP62 reverse primer.
gtcgaataaaaggcgcgcggggcaccaggaagtgg

SEQ ID NO: 121 - DNA, 44 bp, Artificial Sequence
  Description of Artificial Sequence SNP63 foward primer.
acccagtttcaggttttttttttatagcagtgcaagaatgtact

SEQ ID NO: 122 - DNA, 36 bp, Artificial Sequence
  Description of Artificial Sequence SNP63 reverse primer.
gccagggaatagtgtggggattcagagcctgataat

SEQ ID NO: 123 - DNA, 42 bp, Artificial Sequence
  Description of Artificial Sequence SNP64 foward primer.
gcagaattgggcatcactaggcagatgaaacaaagataggat

SEQ ID NO: 124 - DNA, 42 bp, Artificial Sequence
  Description of Artificial Sequence SNP64 reverse primer.
ccacattgagagcttcgactgagtaggcttcctttaaaaagt

SEQ ID NO: 125 - DNA, 44 bp, Artificial Sequence
  Description of Artificial Sequence SNP65 foward primer.
cgcccgaccggatttttttaaagctatgatagataagctcaagg

SEQ ID NO: 126 - DNA, 46 bp, Artificial Sequence
  Description of Artificial Sequence SNP65 reverse primer.
actctaggactaatttacaatcattttcgacattttccttcagcct

SEQ ID NO: 127 - DNA, 44 bp, Artificial Sequence
  Description of Artificial Sequence SNP66 foward primer.
cgtgatgtcaatggagaacttatagctgtgcaaagatctatggt

SEQ ID NO: 128 - DNA, 48 bp, Artificial Sequence
  Description of Artificial Sequence SNP66 reverse primer.
aggaacaatttatcattctttcctacttgtctgcttctcatctcaccc

SEQ ID NO: 129 - DNA, 38 bp, Artificial Sequence
  Description of Artificial Sequence SNP67 foward primer.
tgggccttgaagaatgggaagaaatcaagtcagcaaac

SEQ ID NO: 130 - DNA, 46 bp, Artificial Sequence
  Description of Artificial Sequence SNP67 reverse primer.
agttgcaggtaaaatatactctggaaggttagagatgagaaatgga

SEQ ID NO: 131 - DNA, 42 bp, Artificial Sequence
  Description of Artificial Sequence SNP68 foward primer.
tggcctacttagatctgttttcctttcttgccttaaatggga

SEQ ID NO: 132 - DNA, 36 bp, Artificial Sequence
  Description of Artificial Sequence SNP68 reverse primer.
acaaaaacagccaagggtttaaagcttaggtgccag

SEQ ID NO: 133 - DNA, 36 bp, Artificial Sequence
  Description of Artificial Sequence SNP69 foward primer.
atgtacaggaacaggaatcctcagagtctggagagg

SEQ ID NO: 134 - DNA, 37 bp, Artificial Sequence
  Description of Artificial Sequence SNP69 reverse primer.
tggtgaaagagaccttggcactgctttctactcatcg

SEQ ID NO: 135 - DNA, 46 bp, Artificial Sequence
  Description of Artificial Sequence SNP70 foward primer.
ctgaaaaagaaaagtctggactgaatgagctttcctattctttgat

SEQ ID NO: 136 - DNA, 41 bp, Artificial Sequence
  Description of Artificial Sequence SNP70 reverse primer.
catttgctcctttcaatgaccctatgaggacagtaccatca

SEQ ID NO: 137 - DNA, 46 bp, Artificial Sequence
  Description of Artificial Sequence SNP71 foward primer.
aggtgaggaaggaatagagtgactgctattgaatatgagatttcct

SEQ ID NO: 138 - DNA, 37 bp, Artificial Sequence
  Description of Artificial Sequence SNP71 reverse primer.
ccccaagattctgatttactggtgtggattggagaca

SEQ ID NO: 139 - DNA, 38 bp, Artificial Sequence
  Description of Artificial Sequence SNP72 foward primer.
acctgtagattccttcacactggcttattctccctctg

SEQ ID NO: 140 - DNA, 50 bp, Artificial Sequence
  Description of Artificial Sequence SNP72 reverse primer.
atgagaaaatctaaaatgaatctctgtggataaatcactctggcaacaac

SEQ ID NO: 141 - DNA, 44 bp, Artificial Sequence
  Description of Artificial Sequence SNP73 foward primer.
ccagggaccaagttaaataggcaggttggttttgttttcaattt

SEQ ID NO: 142 - DNA, 43 bp, Artificial Sequence
  Description of Artificial Sequence SNP73 reverse primer.
gcgagcataagccacaaagactcaattttggggaaatttgtat

SEQ ID NO: 143 - DNA, 48 bp, Artificial Sequence
  Description of Artificial Sequence SNP74 foward primer.
ctggtggctttaaaagatggaattttgactgccttttcaattttaact

SEQ ID NO: 144 - DNA, 41 bp, Artificial Sequence
  Description of Artificial Sequence SNP74 reverse primer.
actgtgtaagagatggggatgactaaggtcgctacagtaat

SEQ ID NO: 145 - DNA, 45 bp, Artificial Sequence
  Description of Artificial Sequence SNP75 foward primer.
aggaaactcttaacagggaaactaagaaagagttgaggctgagga

SEQ ID NO: 146 - DNA, 44 bp, Artificial Sequence
  Description of Artificial Sequence SNP75 reverse primer.
atgttggttagcttcaggagagtgtaataatagtagctgagcct

SEQ ID NO: 147 - DNA, 49 bp, Artificial Sequence
  Description of Artificial Sequence SNP76 foward primer.
ctgagttatgaaaattaattagcaataattgtctccttggtgtgagggg

SEQ ID NO: 148 - DNA, 44 bp, Artificial Sequence
  Description of Artificial Sequence SNP76 reverse primer.
aggatacaagacacacagaattgagagagtagggctattctagg

SEQ ID NO: 149 - DNA, 41 bp, Artificial Sequence
  Description of Artificial Sequence SNP77 foward primer.
tccccagctttccacatgtgtcagagaactgtgaggaatga

SEQ ID NO: 150 - DNA, 41 bp, Artificial Sequence
  Description of Artificial Sequence SNP77 reverse primer.
accagtcactagatgcatcattactatgacacacagggacc

SEQ ID NO: 151 - DNA, 35 bp, Artificial Sequence
  Description of Artificial Sequence SNP78 foward primer.
gagaccctgtgggagatgccgtgggccctctacta

SEQ ID NO: 152 - DNA, 38 bp, Artificial Sequence
  Description of Artificial Sequence SNP78 reverse primer.
gatgtcaagatttctcccctacccacttcctccccgaa

SEQ ID NO: 153 - DNA, 40 bp, Artificial Sequence
  Description of Artificial Sequence SNP79 foward primer.
gaagtgggtaggggagaaatcttgacatcaacacccaaca

SEQ ID NO: 154 - DNA, 40 bp, Artificial Sequence
  Description of Artificial Sequence SNP79 reverse primer.
gagagggattgtcaaagttcagatttccaggtctccactg

SEQ ID NO: 155 - DNA, 45 bp, Artificial Sequence
  Description of Artificial Sequence SNP80 foward primer.
acgaaaatttccaatgtaaactcattttccctcggtttcagcaat

SEQ ID NO: 156 - DNA, 42 bp, Artificial Sequence
  Description of Artificial Sequence SNP80 reverse primer.
agcagctgttaacgttttcatgcctagaaatactgagagcat

SEQ ID NO: 157 - DNA, 40 bp, Artificial Sequence
  Description of Artificial Sequence SNP81 foward primer.
actagaatcaggaacgaggagtgactctcagtcagttcat

SEQ ID NO: 158 - DNA, 43 bp, Artificial Sequence
  Description of Artificial Sequence SNP81 reverse primer.
acatatattcaacaaaaatacattcactcatcccaccagccag

SEQ ID NO: 159 - DNA, 38 bp, Artificial Sequence
  Description of Artificial Sequence SNP82 foward primer.
aaacatgatttgaatgctagagcagaaagggccccaga

SEQ ID NO: 160 - DNA, 36 bp, Artificial Sequence
  Description of Artificial Sequence SNP82 reverse primer.
cccttatttcattcctttgcctcttccaacccaagg

SEQ ID NO: 161 - DNA, 46 bp, Artificial Sequence
  Description of Artificial Sequence SNP83 foward primer.
caacatatgaacagtagtatgtacaggcttcaagggttctaattgt

SEQ ID NO: 162 - DNA, 40 bp, Artificial Sequence
  Description of Artificial Sequence SNP83 reverse primer.
gccaagaatcaaatacctggttatgccgtggttgcttatg

SEQ ID NO: 163 - DNA, 39 bp, Artificial Sequence
  Description of Artificial Sequence SNP84 foward primer.
ataagcaaccacggcataaccaggtatttgattcttggc

SEQ ID NO: 164 - DNA, 44 bp, Artificial Sequence
  Description of Artificial Sequence SNP84 reverse primer.
atgctttctttttacatgggctctatttcagttttgataacggt

SEQ ID NO: 165 - DNA, 43 bp, Artificial Sequence
  Description of Artificial Sequence SNP85 foward primer.
cctgcataaaatccacgtgattctgcattctctaatcatacct

SEQ ID NO: 166 - DNA, 44 bp, Artificial Sequence
  Description of Artificial Sequence SNP85 reverse primer.
ggtttcatgacacatttctaatcctgctcagaataagggacacg

SEQ ID NO: 167 - DNA, 41 bp, Artificial Sequence
  Description of Artificial Sequence SNP86 foward primer.
agaaatgagcccaacaggaaccaaagaaagaggaaacaaga

SEQ ID NO: 168 - DNA, 46 bp, Artificial Sequence
  Description of Artificial Sequence SNP86 reverse primer.
ggagggagagactgaaagtggagatagactaacctattatgaaggg

SEQ ID NO: 169 - DNA, 38 bp, Artificial Sequence
  Description of Artificial Sequence SNP87 foward primer.
gaaaagccattccacgggtaggaagacctcatagacat

SEQ ID NO: 170 - DNA, 42 bp, Artificial Sequence
  Description of Artificial Sequence SNP87 reverse primer.
ccattttcagtctgcggtgagaatgaacacaaaaactgagag

SEQ ID NO: 171 - DNA, 37 bp, Artificial Sequence
  Description of Artificial Sequence SNP88 foward primer.
ggaagatggatgcaggcagagctagagaaactgacaa

SEQ ID NO: 172 - DNA, 47 bp, Artificial Sequence
  Description of Artificial Sequence SNP88 reverse primer.
cccatgtgtaaaaaatggcttctggtatgattcatttattttcacgg

SEQ ID NO: 173 - DNA, 40 bp, Artificial Sequence
  Description of Artificial Sequence SNP89 foward primer.
ctgtgaaatcctggaagataaacataacaccgccacacac

SEQ ID NO: 174 - DNA, 36 bp, Artificial Sequence
  Description of Artificial Sequence SNP89 reverse primer.
gtcaacgaggtgtttcggtagtctctggccatcctt

SEQ ID NO: 175 - DNA, 35 bp, Artificial Sequence
  Description of Artificial Sequence SNP90 foward primer.
aaggatggccagagactaccgaaacacctcgttga

SEQ ID NO: 176 - DNA, 37 bp, Artificial Sequence
  Description of Artificial Sequence SNP90 reverse primer.
ggaagttagcaatcaagcgaggtcaggtaggtaaggt

SEQ ID NO: 177 - DNA, 42 bp, Artificial Sequence
  Description of Artificial Sequence SNP91 foward primer.
agatccacccctggtcatagcataaaaagttgaattgtcact

SEQ ID NO: 178 - DNA, 38 bp, Artificial Sequence
  Description of Artificial Sequence SNP91 reverse primer.
gcttttcatgcccctgtcttccttctcatgaccatttc

SEQ ID NO: 179 - DNA, 39 bp, Artificial Sequence
  Description of Artificial Sequence SNP92 foward primer.
tttaccaggccgtattctcaacactgacattcttggtgg

SEQ ID NO: 180 - DNA, 40 bp, Artificial Sequence
  Description of Artificial Sequence SNP92 reverse primer.
cacccagttcccctcattttaatttttctaacctacagct

SEQ ID NO: 181 - DNA, 45 bp, Artificial Sequence
  Description of Artificial Sequence SNP93 foward primer.
aggttagaaaaattaaaatgaggggaactgggtgaattttgcagc

SEQ ID NO: 182 - DNA, 44 bp, Artificial Sequence
  Description of Artificial Sequence SNP93 reverse primer.
ccatcttgctgtcttttgcttctgtttgatttggtctgcatatc

SEQ ID NO: 183 - DNA, 46 bp, Artificial Sequence
  Description of Artificial Sequence SNP94 foward primer.
aggaggagctagaaattaggaattgagagatagaaaatgagcagag

SEQ ID NO: 184 - DNA, 37 bp, Artificial Sequence
  Description of Artificial Sequence SNP94 reverse primer.
tcgatgcgcagtttgaaaattatctgcaggaggagct

SEQ ID NO: 185 - DNA, 45 bp, Artificial Sequence
  Description of Artificial Sequence SNP95 foward primer.
gcatcttgattctccacctattctataagcttctagatgtcggct

SEQ ID NO: 186 - DNA, 35 bp, Artificial Sequence
  Description of Artificial Sequence SNP95 reverse primer.
ggctgcgtgtgtgtgtgtgtgtgtgtgtgtgtgtg

SEQ ID NO: 187 - DNA, 40 bp, Artificial Sequence
  Description of Artificial Sequence SNP96 foward primer.
attgacaaccaatacaaactgagaataccatgagggccca

SEQ ID NO: 188 - DNA, 37 bp, Artificial Sequence
  Description of Artificial Sequence SNP96 reverse primer.
actgggctgcgtaacaaaatgctgttctctactttgt

SEQ ID NO: 189 - DNA, 36 bp, Artificial Sequence
  Description of Artificial Sequence SNP97 foward primer.
ggtctcctccttgccatcctctctgttcctatcatg

SEQ ID NO: 190 - DNA, 35 bp, Artificial Sequence
  Description of Artificial Sequence SNP97 reverse primer.
catctgtcacttccgttgtcacccctgcaatcttg

SEQ ID NO: 191 - DNA, 37 bp, Artificial Sequence
  Description of Artificial Sequence SNP98 foward primer.
gcttgcgagacacctcagcatatttccaaacatgcca

SEQ ID NO: 192 - DNA, 37 bp, Artificial Sequence
  Description of Artificial Sequence SNP98 reverse primer.
gacagcctgataggaatcctagacactccttgttggc

(19) United States
(12) Patent Application Publication
(10) Pub. No.: US 2009/0075285 A1
(43) Pub. Date: Mar. 19, 2009

(54) PRIMER KIT

(75) Inventor: Tetsuya TANABE, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/200,433

(22) Filed: Aug. 28, 2008

(30) Foreign Application Priority Data
Aug. 30, 2007 (JP) 2007-224677

(51) Int. Cl. C12Q 1/68

(52) U.S. Cl. 435/6

(57) ABSTRACT

The aim of the present invention is to provide a primer kit capable of efficiently amplifying only a required target nucleotide sequence without shortage or excess in the case of amplifying a wide range of target nucleotide sequences. The present invention is directed to a primer kit having a plurality of types of primers for amplifying nucleic acids having a plurality of types of target nucleotide sequences, wherein all of the types of primers are primers capable of amplifying each nucleic acid having a target nucleotide sequence under the same reaction conditions, and a single type of primer or primers corresponding to a single target nucleotide sequence is/are housed in at least one container; and a primer kit having a plurality of types of primers for amplifying nucleic acids having a plurality of types of target nucleotide sequences, wherein all of the types of primers are primers capable of amplifying each nucleic acid having a target nucleotide sequence under the same reaction conditions, and primers corresponding to a single target nucleotide sequence are housed in the same container.

### BACKGROUND OF THE INVENTION

1. Field of the Invention

The present invention relates to a primer kit used to amplify nucleic acids having a plurality of types of target nucleotide sequences, and having a plurality of types of primers for which reaction conditions have been standardized.

2. Description of the Related Art

Gene analysis using nucleic acid analysis is mainly carried out by recognizing a characteristic nucleotide sequence and detecting whether or not a nucleic acid having that nucleotide sequence is present in a specimen. This type of gene analysis is widely used in numerous fields such as medicine, scientific research and industry, as a result of progress in the areas of gene manipulation technology and gene recombination technology. For example, this type of gene analysis is applied to the diagnosis and treatment of diseases such as genetic diseases, cancer, infectious diseases and lifestyle diseases, and is also used in the testing of foods such as meats and grains.

In cases when there is only an extremely small amount of specimen or in cases in which the concentration of nucleic acids in a specimen is extremely low, as is the case with specimens for clinical laboratory testing, analyses are frequently carried out after obtaining first amplified a nucleic acid having the target nucleotide sequence to be analyzed. The polymerase chain reaction (PCR) method is most commonly used to amplify such nucleotide sequences. The PCR method uses two types of primers on both sides of a target nucleotide sequence to be analyzed and DNA polymerase to exponentially amplify the target nucleotide sequence. The PCR method basically consists of: (1) a denaturation step, (2) an annealing step, and (3) an elongation step for one cycle, and by repeating that cycle, a specific target nucleotide sequence is amplified (refer to, for example, Patent Document 1). PCR can be used to selectively amplify a nucleic acid having a target nucleotide sequence to a detectable label in several cycles using heat-resistant polymerase in the presence of deoxynucleotide triphosphate.

In this type of PCR method, it is preferable to use primers that are unlikely to form primer dimmers and the like caused by the primers annealing to nucleotide sequences other than the target nucleotide sequence or annealing between primers in order to prevent non-specific nucleic acid amplification. Consequently, primer design is extremely important, and numerous software has been developed for primer design. However, even in the case of using such primer design software, the design success rate, namely the probability of being able to design primers that allow a target nucleic acid to be selectively and efficiently amplified while preventing non-specific nucleic acid amplification, is roughly about 70%, and it is necessary to optimize the reaction conditions with respect to salt concentration, reaction temperature, enzyme concentration and the like in order to improve amplification efficiency. As a result, conventional PCR kits cannot be intermixed since the reaction conditions are optimized for each target nucleotide sequence, and in the case of multiple target nucleotide sequences, it was necessary to carry out PCR separately for each target nucleotide sequence.

On the other hand, in clinical laboratory testing for diagnosing a disease, for example, although there are tests requiring measurement of numerous specimens, such as in testing for drug metabolic capacity, there are also many tests for which the need for measurement varies considerably for each specimen, such as when detecting a specific disease-related factor. Consequently, tests required for each specimen are typically different. In the case of genetic testing in particular, since target nucleotide sequences differ for each test, it is necessary to carry out PCR separately for a number of times equal to the number of tests on a single specimen, thereby making it difficult to reduce costs and improve throughput.

In such cases, throughput can be improved by applying multiplex PCR. Multiplex PCR differs from ordinary PCR in that amplification is carried out on a plurality of types of target nucleotide sequences by using a plurality of types of primer sets in a single reaction container. The use of this technique makes it possible to carry out PCR required for a large number of types of tests only once.

[Patent Document 1] Japanese Examined Patent Application, Second Publication No. H4-67960

Normally, in kits having primer groups for carrying out multiplex PCR on a plurality of types of target nucleotide sequences, primer design and reaction conditions are optimized for the purpose of satisfactory amplifying each nucleic acid having a target nucleotide sequence in the case of carrying out PCR using these primer groups in a single reaction solution. In the case of conventional multiplex PCR in particular, there are many kits that attempt to maintain a uniform amplification efficiency by adjusting the concentration of each primer, thus making it extremely difficult to freely select those primers required for use from among the primer groups present in a multiplex PCR kit. Consequently, despite the genetic information having come to be treated as important personal information in recent years, there was the problem of multiplex PCR kits ending up detecting unnecessary genetic information in cases in which primers for amplifying target nucleotide sequences not required for testing are contained in the primer groups of multiplex PCR kits.

In addition, in the case of multiplex PCR, the number of each PCR product tends to decrease corresponding to the number of target nucleotide sequences to be amplified, and in cases in which the PCR product measurement method is low, an adequate amount of PCR product for detection may not be able to be obtained depending on the number of target nucleotide sequences, thereby resulting in the problem of difficulties in carrying out testing.

An object of the present invention is to provide a primer kit capable of efficiently amplifying only a required target nucleotide sequence without shortage or excess, improving throughput and reducing costs in cases of amplifying a diverse range of target nucleotide sequences.

### SUMMARY OF THE INVENTION

As a result of conducting extensive studies to solve the problems described above, the inventors of the present invention found that, if reaction conditions are standardized for all of the primers that compose a primer group capable of being used in multiplex PCR, and if the primers are suitably stored separately so that they can be discarded or selected as necessary, primers required for each specimen can be used interchangeably, thereby making it possible to avoid detection of unnecessary genetic information, and even in the case of using any combination of primers, since amplification reactions can be carried out under identical conditions, costs can be decreased and throughput can be improved, thereby leading to completion of the present invention.

Namely, the present invention provides a primer kit having a plurality of types of primers for amplifying nucleic acids having a plurality of types of target nucleotide sequences; wherein, all of the types of primers are primers capable of amplifying each nucleic acid having a target nucleotide sequence under the same reaction conditions, and a single type of primer or primers corresponding to a single target nucleotide sequence is/are housed in at least one container.

In addition, the present invention provides a primer kit in which primers corresponding to a single target nucleotide sequence are housed in a single container.

In addition, the present invention provides a primer kit in which a single type of primer is housed in a single container.

In addition, the present invention provides a primer kit having a plurality of types of primers for amplifying nucleic acids having a plurality of types of target nucleotide sequences; wherein, all of the types of primers are primers capable of amplifying each nucleic acid having a target nucleotide sequence under the same reaction conditions, and primers corresponding to a single target nucleotide sequence are housed in the same container.

In addition, the present invention provides a primer kit in which amplification is carried out by polymerase chain reaction (PCR).

In addition, the present invention provides a primer kit in which the base length of the primers is 30 bases or more.

In addition, the present invention provides a primer kit in which the reaction conditions are such that the sum of annealing time and elongation reaction time is 3 minutes or more.

In addition, the present invention provides a primer kit in which the reaction conditions are such that denaturation temperature, annealing temperature and elongation reaction temperature are all 68° C. or higher.

In addition, the present invention provides a primer kit in which the reaction conditions are such that the annealing temperature and the elongation reaction temperature are the same.

In addition, the present invention provides any of the primer kits described above containing heat-resistant DNA polymerase.

In addition, the present invention provides any of the primer kits described above containing a reaction buffer.

In addition, the present invention provides a primer kit that is provided with a table containing target nucleotide sequences and combinations of primers used to amplify nucleic acids having those target nucleotide sequences.

### BRIEF DESCRIPTION OF THE DRAWINGS

FIG. 1 shows band patterns of reaction solutions following PCR amplification as confirmed by electrophoresis in Example 1. FIG. 1(A) shows the band patterns obtained by agarose gel electrophoresis, and the values shown above the lanes indicate the elongation time (minutes) of each reaction solution. In addition, the lanes on both ends in the figure indicate the results of electrophoresing base pair length markers. FIG. 1(B) shows the band pattern of a reaction solution using an elongation time of 6 minutes as confirmed with a Model 2100 Bioanalyzer with band fluorescence intensity plotted on the vertical axis and phoresis time (seconds) plotted on the horizontal axis. Arrow (a) indicates the band for SNP3, arrow (b) SNP4, arrow (c) SNP5, arrow (d) SNP6, arrow (e) SNP7, arrow (f) SNP8, arrow (g) SNP9, arrow (h) SNP10, arrow (i) SNP11 and arrow (j) SNP12.

FIG. 2 shows the band patterns of reaction solutions following PCR amplification of 96 types of primer sets obtained by staining with ethidium bromide following agarose gel electrophoresis in Example 2. The lanes on both ends of all four rows indicate the results of electrophoresing base pair length markers.

FIG. 3 shows the band patterns of reaction solutions following PCR amplification as confirmed with a Model 2100 Bioanalyzer. FIG. 3(A) shows the band patterns of the reaction solutions to which sets A to L were added, while FIG. 3(B) shows the band patterns of the reaction solutions to which sets M to X were added. In the figures, the lane on the left end of the band patterns indicates the results of electrophoresing a base pair length marker. In addition, the bands for 1500 bp and 150 bp present in all lanes indicate the patterns for the phoretic dye.

### DETAILED DESCRIPTION OF THE PREFERRED EMBODIMENTS

Use of the primer kit of the present invention makes it possible to interchangeably use primers required for each specimen as desired. Consequently, during genetic testing and the like in which different tests are required for each specimen, only required genetic information can be amplified without shortage or excess while avoiding detection of unnecessary genetic information. In addition, since the reaction conditions of each primer are standardized, each target nucleotide sequence can be efficiently amplified under the same reaction conditions regardless of which combination of primers is used. Thus, the use of the primer kit of the present invention makes it possible to realize reduced costs and improved throughput for overall testing by allowing primer sets to be interchanged as necessary and carrying out testing under the same reaction conditions even in cases in which testing is performed on a plurality of specimens requiring different tests.

A target nucleotide sequence as referred to in the present invention indicates a nucleotide sequence targeted for an amplification reaction, and there are no particular limitations thereon provided the nucleotide sequence has been clearly identified to an extent that allows amplification by gene recombination technology and the like. For example, the target nucleotide sequence may be a nucleotide sequence present in animal or plant chromosomes or present in bacterial or viral genes, and may also be a nucleotide sequence present in mRNA or other RNA of living organisms.

There are no particular limitations on the nucleic acid having a target nucleotide sequence as referred to in the present invention (to be referred to as a target nucleic acid) provided it has a target nucleotide sequence and is able to serve as a template in nucleic acid amplification. For example, the nucleic acid may be a nucleic acid contained in a biological sample (specimen) such as blood or body fluid, a nucleic acid extracted from these biological samples and the like, or a nucleic acid amplified by using these nucleic acids as templates. In addition, the nucleic acid may also be cDNA synthesized using reverse transcriptase from RNA contained in a biological sample.

In the present invention, there are no particular limitations on the method used to amplify nucleic acid provided it uses hybridization between primers and a target nucleic acid, and various nucleic acid amplification methods ordinarily used in fields such as gene analysis can be used. The primer kit of the present invention is preferably a primer kit for amplifying nucleic acids by PCR to ensure high accuracy and universality of the nucleic acid amplification.

The primer kit of the present invention is a primer kit having a plurality of types of primers for amplifying a plurality of types of target nucleic acids, all of the types of primers are primers capable of amplifying each target nucleic acid under the same reaction conditions, and a single type of primer or primers corresponding to a single target nucleotide sequence is/are contained in at least one container. All of the primers contained in the primer kit are not stored in a mixed state, but rather as a result of storing all or a portion of the types of primers separately according to container, primers can be selected from the group of primers for use in an amplification reaction.

Although primers are housed in a container in the primer kit of the present invention, there are no particular limitations on the container provided there is at least one container in which a single type of primer or primers corresponding to a single target nucleotide sequence are housed, and the primers can be suitably housed in the container in consideration of, for example, the frequency at which each combination of primers is used. Here, “primers corresponding to a single target nucleotide sequence” refer to those primers required to amplify a single target nucleotide sequence that are capable of hybridizing with a nucleic acid having the target nucleotide sequence or nucleic acid having a nucleotide sequence complementary to the target nucleotide sequence. More specifically, these primers refer to forward and reverse primers capable of amplifying a nucleic acid by PCR in the case of carrying out amplification of a target nucleic acid by PCR.

For example, primers corresponding to a single target nucleotide sequence or a single type of primer may be housed in one of all of the containers of the primer kit of the present invention. In the case of housing primers corresponding to a single target nucleotide sequence in a single container, the procedure can be simplified since primers for amplifying each target nucleic acid can be added to a reaction solution with a single dispensing procedure. On the other hand, in the case of housing a single type of primer in a single container, the degree of freedom when combining primers can be maximized.

For example, in the case primers corresponding to a target nucleotide sequence N₁ consist of a forward primer F₁ and a reverse primer R₁, primers corresponding to a target nucleotide sequence N₂ consist of a forward primer F₂ and a reverse primer R₂, primers corresponding to a target nucleotide sequence N₃ consist of forward primer F₃ and reverse primer R₃, and primers corresponding to a target nucleotide sequence N₄ consist of a forward primer F₄ and a reverse primer R₄, forward primer F₁ and reverse primer R₁ are housed in one container, forward primer F₂ and reverse primer R₂ are housed in another container, forward primer F₃ and reverse primer R₃ are housed in still another container, and forward primer F₄ and reverse primer R₄ are housed in yet still another container.

In addition, in the case primers corresponding to a target nucleotide sequence N₁ consist of a forward primer F₁ and a reverse primer R₁, primers corresponding to a target nucleotide sequence N₂ consist of a forward primer F₁ and a reverse primer R₂, primers corresponding to a target nucleotide sequence N₃ consist of forward primer F₃ and reverse primer R₂, and primers corresponding to a target nucleotide sequence N₄ consist of a forward primer F₁ and a reverse primer R₄, each primer can be respectively housed in 8 separate containers.

The primer kit of the present invention is a primer kit having a plurality of types of primers for amplifying a plurality of types of target nucleic acids, all of the types of primers are primers capable of amplifying each target nucleic acid sequence under the same reaction conditions, and primers corresponding to a single target nucleotide sequence are housed in the same container. For example, in the case target nucleotide sequences N₁ and N₂ are required to be detected in a large number of specimens, while target nucleotide sequences N₃ and N₄ have different degrees of being required to be detected for each specimen, by housing primers corresponding to target nucleotide sequence N₁ and primers corresponding to target nucleotide sequence N₂ in a single container, housing primers corresponding to target nucleotide sequence N₃ in another container, and housing primers corresponding to target nucleotide sequence N₄ in still another container, the complexity of the dispensing procedure can be reduced while maintaining the degree of freedom in selecting the required primers. In addition, in the case the frequency of simultaneously detecting target nucleotide sequences N₁ and N₂ is high, and the frequency of detecting target nucleotide sequences N₃ and N₄ is also high, primers corresponding to target nucleotide sequence N₁ and primers corresponding to target nucleotide sequence N₂ may be housed in a single container, and primers corresponding to target nucleotide sequence N₃ and primers corresponding to target nucleotide sequence N₄ may be housed in another single container.

In the present invention, there are no particular limitations on the containers provided they are containers capable of housing and storing nucleic acids separately, and examples thereof include containers such as 0.6 mL tubes and 1.5 mL tubes ordinarily used to store nucleic acids in fields such as genetic recombination technology. In addition, each container is only required to be able to maintain the housed primers in a separated state, although each container is not required to be a separate, independent container. For example, a capped 8-strip PCR tube or capped 12-strip PCR tube may be used.

In the present invention, primers housed in the containers may be freeze-dried, or may be dissolved in a suitable buffer such as ultra-pure water or tris-EDTA (TE) buffer.

The primers that compose the primer kit of the present invention are all capable of amplifying each nucleic acid having a target nucleotide sequence under the same reaction conditions. Here, reaction conditions refer to various conditions in the amplification reaction of a target nucleic acid, such as the amounts of specimen and primers added to the reaction solution, the composition of the reaction buffer, the reaction temperature and the reaction time. For example, in the case of carrying out amplification of a target nucleic acid by PCR, this means that conditions, such as the salt composition and other parameters of the composition of the reaction buffer, the type of heat-resistant DNA polymerase, the added amounts of specimen and primers, the temperatures and times n each of the denaturation, annealing and elongation steps, and the number of cycles, are the same.

Here, the added amount of primers refers to the total amount of all types of primers added to the reaction solution. In other words, in the case, for example, the amount of primer added is 10 μM and there are two types of primers added, then the amount of each primer is 5 μM, while in the case of adding ten types of primers, the amount of each primer added becomes 1 μM.

Namely, the primers that compose the primer kit of the present invention are capable of amplifying a target nucleic acid present in a reaction solution regardless of the types of primers simultaneously added to the reaction solution. Consequently, even in the case only required primers are freely selected from a plurality of types of primers present in the primer kit of the present invention, the primers are able to efficiently amplify each target nucleic acid without having to change the reaction conditions.

The primer kit of the present invention may be used to carry out multiplex PCR or ordinary PCR. For example, in the case of amplifying in order to detect and measure six types of target nucleic acids, and the sensitivity of the PCR product measurement method is low, an amount of PCR product sufficient for measurement can be obtained by carrying out PCR reactions in separate reaction solutions for each target nucleic acid. On the other hand, in the case the sensitivity of the PCR product measurement method is high or the amount of specimen is extremely low, multiplex PCR may be carried out by adding all primers corresponding to the six types of target nucleic acids to a single reaction solution.

A primer group capable of amplifying each target nucleic acid under the same reaction conditions in this manner can be obtained by standardizing the reaction conditions of each primer. For example, since reaction conditions are greatly affected by annealing efficiency between the primers and the target nucleic acids serving as templates in PCR, by making the annealing efficiency of each primer nearly uniform, reaction conditions of nucleic acid amplification can be standardized (see, for example, Japanese Unexamined Patent Application, First Publication No. 2006-320217).

More specifically, annealing efficiency, or in other words hybridization efficiency, can be made to be nearly uniform at 90% or more by designing primers to have a region that hybridizes with a target nucleic acid of the primers of 30 bases or more and have a Tm value of 70 to 100° C., and by making the reaction conditions to be such that the sum of annealing time and elongation reaction time in particular is 3 minutes or more. Under such conditions, there is an extremely high possibility of being able to design primers capable of amplifying target nucleic acids under uniform reaction conditions less susceptible to the effects of the number of target nucleic acids or the secondary structures of the target nucleic acids. Since primer specificity is improved by increasing the base length, cross-hybridization is inhibited, thereby enabling target nucleic acids to amplified with high specificity. The base length of the region that hybridizes with the target nucleic acids of the primers is preferably 30 to 60 bases, more preferably 32 to 50 bases, and particularly preferably 35 to 45 bases.

The Tm value of a primer typically increases as base length becomes longer. Consequently, although there are many cases in which primer Tm value is increased by the elongation temperature as a result of making the base length 30 bases or more, the annealing temperature is preferably about the same as the elongation temperature. Even if the elongation temperature is significantly lower than the Tm value, by annealing at about the same temperature as the elongation temperature, it is presumed that annealing efficiency can be improved regardless of the type of target nucleic acid. It is particularly preferable to make the annealing temperature and the elongation temperature equal, and allow annealing and the elongation reaction to proceed simultaneously in the form of shuttle PCR. Furthermore, the elongation temperature can be suitably determined in consideration of such factors as the type of heat-resistant polymerase used.

In addition, by making the sum of annealing time and elongation reaction time three minutes or more, the efficiency by which target nucleic acids are amplified can be increased even if using primers having a base length of 30 bases or more. Increasing the sum of annealing time and elongation reaction time is presumed to ensure adequate times required for accurate annealing and elongation even in cases of using long primers. The sum of annealing time and elongation reaction time is preferably 3 to 10 minutes, more preferably 5 to 10 minutes, even more preferably 5 to 8 minutes, and particularly preferably about 6 minutes. Moreover, due to the long elongation time, amplification can be carried out adequately even in the case of target nucleic acids having long base pair lengths.

In addition, the denaturation step and setting of the number of amplification cycles can be carried out in the same manner as ordinary PCR. In addition, there are no particular limitations on the reagents used for PCR, such as the heat-resistant polymerase, nucleotides and reaction buffer, and those ordinarily used for carrying out PCR can be used in the normally used amounts. There are also no particular limitations on the specimen and primers provided they are used in amounts normally used.

Such primers can be designed using any method known in the relevant technical field. For example, primers can be easily designed by using known genome sequence data and commonly used primer design tools. Examples of such a primer design tools include Primer 3 able to be utilized online (Rozen, S., H. J. Skaletsky, 1996, http://www-genome.wi.mit+edu/genome_software/otherprimer3.html) and Visual OMP (DNA Software, Inc.). In particular, annealing efficiency can be easily measured by inputting, for example, the nucleic acid concentration of the reaction solution, salt concentration, and temperature. In the case of obtaining a plurality of primer candidates by using such primer design tools, it is preferably to select primers having high annealing efficiency in consideration of the primers and other parameters such as the predicted secondary structures of the target nucleic acid. In addition, known genome sequence data can normally be easily acquired from international nucleotide sequence databases such as the National Center for Biotechnology Information (NCBI) or the DNA Data Bank of Japan (DDBJ).

Primers designed in this manner can be synthesized using any method known in the relevant technical field. For example, the primers may be synthesized by commissioning synthesis to an manufacturer engaged in the synthesis of oligonucleotides or by synthesizing in-house using a commercially available synthesis system. In addition, each primer can have additional sequences to a degree that does not impair amplification of the target nucleic acid in addition to the region that hybridizes with the target nucleic acid. Examples of such additional sequences include restriction enzyme recognition sequences and sequences provided for labeling nucleic acids.

In other words, by making primer annealing efficiency nearly uniform and inhibiting variations in amplification efficiency, a primer group can be prepared that not only allows application to multiplex PCR for simultaneously amplifying a plurality of types of target nucleic acids, but also permits primer sets to be interchanged. Since amplification efficiency of primer sets within the primer group does not depend on their combination, the primer set used can be interchanged as necessary. Moreover, there is no need to adjust the reaction conditions regardless of the combination of primer sets used, and all primer sets can be used under the same conditions.

In addition, primers that compose the primer kit of the present invention may be labeled to facilitate detection and analysis of the amplified target nucleic acid. There are no particular limitations on the substances used for labeling provided they can be used to label nucleic acids, and examples of such substances include radioisotopes, fluorescent substances, chemiluminescent substances and biotin. For example, even in the case of amplifying a plurality of target nucleic acids having identical base pair lengths, the use of primers modified by using a label makes it possible to identify the target nucleic acids.

In addition, the primer kit of the present invention may also have reagents and the like used to amplify nucleic acids in addition to primers. For example, the primer kit preferably has a heat-resistant polymerase, and may also have a reaction buffer. In addition, a table containing target nucleotide sequences and combinations of primers used to amplify nucleic acids having those target nucleotide sequences is more preferably provided.

### EXAMPLES

Although the following provides a more detailed explanation of the present invention by indicating examples thereof, the present invention is not limited to the following examples. Furthermore, the SNP nucleotide sequences used were obtained from the Japanese SNP Database located at the Institute of Medical Science of The University of Tokyo (http://snp.ims.u-tokyo.ac.jp/index_ja.html).

### Example 1

Human genome nucleotide sequences possessed by Japanese SNP 3 to 12 shown in Table 1 were attempted to be amplified using these nucleotide sequences as target nucleotide sequences. Their respective accession numbers are shown in Table 1.

TABLE 1

| | Accession No. |
|---|---|
| SNP3 | IMS-JST164838 |
| SNP4 | IMS-JST058048 |
| SNP5 | IMS-JST005689 |
| SNP6 | IMS-JST054229 |
| SNP7 | IMS-JST001164 |
| SNP8 | IMS-JST017558 |
| SNP9 | IMS-JST175404 |
| SNP10 | IMS-JST054214 |
| SNP11 | IMS-JST011815 |
| SNP12 | IMS-JST156026 |

First, a primer kit was prepared having primer sets P3 to P12 composed of forward primers and reverse primers for amplifying each target nucleotide sequence SNP3 to SNP12 by PCR. Each primer of the primer kit was designed using Visual OMP to have a base length of 30 bases or more, a Tm value of 70 to 100° C. and an annealing efficiency of 90% or more. The resulting primers are shown in Table 2. Furthermore, in Table 2, “bp” indicates the base pair length of the target nucleic acids targeted for PCR amplification, while “Fw” and “Rv” respectively indicate the forward primer (Fw) and reverse primer (Rv) used to amplify target nucleotide sequences containing each SNP by PCR.

TABLE 2

| | bp | Fw | Rv |
|---|---|---|---|
| P3 | 256 | SEQ. ID. | SEQ. ID. |
| | | NO. 1 | NO. 2 |
| P4 | 352 | SEQ. ID. | SEQ. ID. |
| | | NO. 3 | NO. 4 |
| P5 | 701 | SEQ. ID. | SEQ. ID. |
| | | NO. 5 | NO. 6 |
| P6 | 791 | SEQ. ID. | SEQ. ID. |
| | | NO. 7 | NO. 8 |
| P7 | 684 | SEQ. ID. | SEQ. ID. |
| | | NO. 9 | NO. 10 |
| P8 | 311 | SEQ. ID. | SEQ. ID. |
| | | NO. 11 | NO. 12 |
| P9 | 475 | SEQ. ID. | SEQ. ID. |
| | | NO. 13 | NO. 14 |
| P10 | 413 | SEQ. ID. | SEQ. ID. |
| | | NO. 15 | NO. 16 |
| P11 | 799 | SEQ. ID. | SEQ. ID. |
| | | NO. 17 | NO. 18 |
| P1 | 424 | SEQ. ID. | SEQ. ID. |
| | | NO. 19 | NO. 20 |

Primer sets P3 to P12 were each added to a single reaction solution, and shuttle PCR was carried out using various elongation times. Human genome, Genome Mix (Novagen), was used for the specimen.

More specifically, 5 ng of Genome Mix and all primer types of primer sets P3 to P12 were respectively added to 10 μL of 2× AccuPrime II Master Mix (Invitrogen) to final concentrations of 0.1 μM to prepare 20 μL of each reaction solution. After treating the reaction solutions for 2 minutes at 94° C., PCR was carried out by carrying out 40 heat cycles, each consisting of 30 seconds at 94° C. and 30 seconds to 8 minutes at 68° C., followed finally by carrying out an elongation reaction for 10 minutes at 68° C. Subsequently, 1 μL aliquots of the resulting reaction solutions were recovered followed by confirming in electrophoresis whether or not the 10 types of target nucleic acids were amplified.

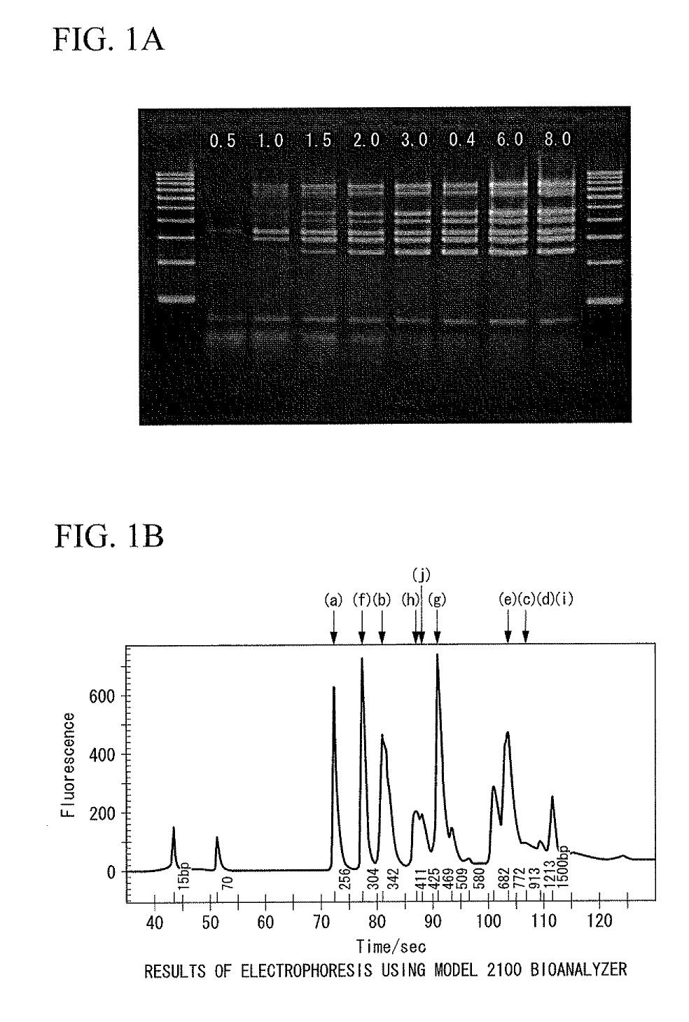

FIG. 1 shows the band patterns of the reaction solutions following PCR amplification as confirmed by electrophoresis. FIG. 1(A) shows the band patterns obtained by agarose gel electrophoresis, and the values shown above the lanes indicate the elongation time (minutes) of each reaction solution. In addition, the lanes on both ends in the figure indicate the results of electrophoresing base pair length markers. FIG. 1(B) shows the band pattern of a reaction solution using an elongation time of 6 minutes as confirmed with the Model 2100 Bioanalyzer (Agilnet) with band fluorescence intensity plotted on the vertical axis and phoresis time (seconds) plotted on the horizontal axis. Arrow (a) indicates the band for SNP3, arrow (b) SNP4, arrow (c) SNP5, arrow (d) SNP6, arrow (e) SNP7, arrow (f) SNP8, arrow (g) SNP9, arrow (h) SNP10, arrow (i) SNP11 and arrow (j) SNP12. Although amplification of several target nucleic acids was unable to be confirmed when the elongation time was 1.5 minutes or shorter, all ten types of target nucleic acids were able to be confirmed to have been amplified when the elongation time was 3 minutes or longer. In other words, on the basis of the results of Example 17 as a result of carrying out multiplex PCR using a primer set for which reaction conditions had been standardized so that base pair length was 30 bases or more, Tm values were 70 to 100° C. and annealing efficiency was 90% or more, all target nucleic acids were clearly demonstrated to be able to be satisfactorily amplified.

### Example 2

SNP3 to SNP12 used in Example 1 along with an additional 86 types of SNP (SNP13 to SNP98) were attempted to be amplified by using 96 sites in the human genome as target nucleotide sequences. Furthermore, the accession numbers of SNP13 to SNP98 are shown in Table 3.

TABLE 3

| | Accession No. |
|---|---|
| SNP13 | ssj0008397 |
| SNP14 | IMS-JST150334 |
| SNP15 | IMS-JST150336 |
| SNP16 | IMS-JST150338 |
| SNP17 | IMS-JST164830 |
| SNP18 | IMS-JST150341 |
| SNP19 | IMS-JST164833 |
| SNP20 | IMS-JST000452 |
| SNP21 | ssj0008401 |
| SNP22 | IMS-JST000454 |
| SNP23 | ssj0005226 |
| SNP24 | ssj0005227 |
| SNP25 | IMS-JST190204 |
| SNP26 | IMS-JST150345 |
| SNP27 | IMS-JST150346 |
| SNP28 | IMS-JST115271 |
| SNP29 | IMS-JST164836 |
| SNP30 | IMS-JST164839 |
| SNP31 | IMS-JST150350 |
| SNP32 | ssj0008415 |
| SNP33 | IMS-JST164842 |
| SNP34 | ssj0008419 |
| SNP35 | ssj0005239 |
| SNP36 | IMS-JST150353 |
| SNP37 | IMS-JST064480 |
| SNP38 | ssj0008424 |
| SNP39 | ssj0005245 |
| SNP40 | ssj0005247 |
| SNP41 | ssj0008429 |
| SNP42 | ssj0008431 |
| SNP43 | ssj0005249 |
| SNP44 | ssj0008433 |
| SNP45 | IMS-JST000080 |
| SNP46 | ssj0008437 |
| SNP47 | ssj0005253 |
| SNP48 | IMS-JST101643 |
| SNP49 | ssj0005254 |
| SNP50 | IMS-JST000084 |
| SNP51 | ssj0008447 |
| SNP52 | ssj0008448 |
| SNP53 | IMS-JST000086 |
| SNP54 | IMS-JST001553 |
| SNP55 | ssj0008452 |
| SNP56 | IMS-JST035951 |
| SNP57 | IMS-JST024135 |
| SNP58 | IMS-JST101727 |
| SNP59 | IMS-JST005692 |
| SNP60 | IMS-JST005685 |
| SNP61 | IMS-JST005684 |
| SNP62 | IMS-JST005683 |
| SNP63 | IMS-JST054230 |
| SNP64 | IMS-JST054228 |
| SNP65 | IMS-JST054227 |
| SNP66 | IMS-JST116906 |
| SNP67 | IMS-JST054226 |
| SNP68 | IMS-JST032051 |
| SNP69 | rs2069822 |
| SNP70 | IMS-JST087914 |
| SNP71 | IMS-JST054223 |
| SNP72 | IMS-JST057868 |
| SNP73 | IMS-JST156275 |
| SNP74 | IMS-JST017560 |
| SNP75 | IMS-JST001165 |
| SNP76 | IMS-JST007962 |
| SNP77 | IMS-JST011440 |
| SNP78 | IMS-JST109935 |
| SNP79 | IMS-JST133234 |
| SNP80 | IMS-JST005923 |
| SNP81 | IMS-JST054219 |
| SNP82 | IMS-JST109933 |
| SNP83 | IMS-JST156272 |
| SNP84 | IMS-JST156270 |
| SNP85 | IMS-JST017555 |
| SNP86 | IMS-JST054213 |
| SNP87 | IMS-JST054212 |
| SNP88 | IMS-JST156269 |
| SNP89 | IMS-JST073854 |
| SNP90 | IMS-JST109931 |
| SNP91 | IMS-JST024230 |
| SNP92 | IMS-JST036418 |
| SNP93 | IMS-JST036416 |
| SN294 | IMS-JST011813 |
| SNP95 | IMS-JST011811 |
| SNP96 | IMS-JST024279 |
| SNP97 | IMS-JST011807 |
| SNP98 | IMS-JST011805 |

Primer sets P13 to P98, composed of forward primers and reverse primers for amplifying each target nucleotide sequence of SNP13 to SNP98 by PCR, were prepared, and added to primer sets P3 to P12 used in Example 1 to prepare a primer kit having primer sets P3 to P98. Each of the primers of primer sets P13 to P98 was designed using VISUAL OMP to have a base length of 30 bases or more, a Tm value of 70 to 100° C. and annealing efficiency of 90% or more in the same manner as Example 1. The resulting primers are shown in Tables 4 and 5.

Furthermore, “bp”, “Fw” and “Rv” shown in Tables 4 and 5 are the same as previously defined for Table 2.

TABLE 4

| | bp | Fw | Rv |
|---|---|---|---|
| P13 | 382 | SEQ. ID. | SEQ. ID. |
| | | NO. 21 | NO. 22 |
| P14 | 792 | SEQ. ID. | SEQ. ID. |
| | | NO. 23 | NO. 24 |
| P15 | 669 | SEQ. ID. | SEQ. ID. |
| | | NO. 25 | NO. 26 |
| P16 | 739 | SEQ. ID. | SEQ. ID. |
| | | NO. 27 | NO. 28 |
| P17 | 632 | SEQ. ID. | SEQ. ID. |
| | | NO. 29 | NO. 30 |
| P18 | 544 | SEQ. ID. | SEQ. ID. |
| | | NO. 31 | NO. 32 |
| P19 | 549 | SEQ. ID. | SEQ. ID. |
| | | NO. 33 | NO. 34 |
| P20 | 777 | SEQ. ID. | SEQ. ID. |
| | | NO. 35 | NO. 36 |
| P21 | 462 | SEQ. ID. | SEQ. ID. |
| | | NO. 37 | NO. 38 |
| P22 | 510 | SEQ. ID. | SEQ. ID. |
| | | NO. 39 | NO. 40 |
| P23 | 539 | SEQ. ID. | SEQ. ID. |
| | | NO. 41 | NO. 42 |
| P24 | 673 | SEQ. ID. | SEQ. ID. |
| | | NO. 43 | NO. 44 |
| P25 | 439 | SEQ. ID. | SEQ. ID. |
| | | NO. 45 | NO. 46 |
| P26 | 358 | SEQ. ID. | SEQ. ID. |
| | | NO. 47 | NO. 48 |
| P27 | 524 | SEQ. ID. | SEQ. ID. |
| | | NO. 49 | NO. 50 |
| P28 | 249 | SEQ. ID. | SEQ. ID. |
| | | NO. 51 | NO. 52 |
| P29 | 572 | SEQ. ID. | SEQ. ID. |
| | | NO. 53 | NO. 54 |
| P30 | 579 | SEQ. ID. | SEQ. ID. |
| | | NO. 55 | NO. 56 |
| P31 | 277 | SEQ. ID. | SEQ. ID. |
| | | NO. 57 | NO. 58 |
| P32 | 530 | SEQ. ID. | SEQ. ID. |
| | | NO. 59 | NO. 60 |
| P33 | 234 | SEQ. ID. | SEQ. ID. |
| | | NO. 61 | NO. 62 |
| P34 | 259 | SEQ. ID. | SEQ. ID. |
| | | NO. 63 | NO. 64 |
| P35 | 381 | SEQ. ID. | SEQ. ID. |
| | | NO. 65 | NO. 66 |
| P36 | 437 | SEQ. ID. | SEQ. ID. |
| | | NO. 67 | NO. 68 |
| P37 | 551 | SEQ. ID. | SEQ. ID. |
| | | NO. 69 | NO. 70 |
| P38 | 652 | SEQ. ID. | SEQ. ID. |
| | | NO. 71 | NO. 72 |
| P39 | 651 | SEQ. ID. | SEQ. ID. |
| | | NO. 73 | NO. 74 |
| P40 | 338 | SEQ. ID. | SEQ. ID. |
| | | NO. 75 | NO. 76 |
| P41 | 181 | SEQ. ID. | SEQ. ID. |
| | | NO. 77 | NO. 78 |
| P42 | 703 | SEQ. ID. | SEQ. ID. |
| | | NO. 79 | NO. 80 |
| P43 | 251 | SEQ. ID. | SEQ. ID. |
| | | NO. 81 | NO. 82 |
| P44 | 508 | SEQ. ID. | SEQ. ID. |
| | | NO. 83 | NO. 84 |
| P45 | 391 | SEQ. ID. | SEQ. ID. |
| | | NO. 85 | NO. 86 |
| P46 | 536 | SEQ. ID. | SEQ. ID. |
| | | NO. 87 | NO. 88 |
| P47 | 665 | SEQ. ID. | SEQ. ID. |
| | | NO. 89 | NO. 90 |
| P48 | 356 | SEQ. ID. | SEQ. ID. |
| | | NO. 91 | NO. 92 |
| P49 | 306 | SEQ. ID. | SEQ. ID. |
| | | NO. 93 | NO. 94 |
| P50 | 698 | SEQ. ID. | SEQ. ID. |
| | | NO. 95 | NO. 96 |
| P51 | 523 | SEQ. ID. | SEQ. ID. |
| | | NO. 97 | NO. 98 |
| P52 | 742 | SEQ. ID. | SEQ. ID. |
| | | NO. 99 | NO. 100 |
| P53 | 790 | SEQ. ID. | SEQ. ID. |
| | | NO. 101 | NO. 102 |
| P54 | 373 | SEQ. ID. | SEQ. ID. |
| | | NO. 103 | NO. 104 |
| P55 | 620 | SEQ. ID. | SEQ. ID. |
| | | NO. 105 | NO. 106 |
| P56 | 426 | SEQ. ID. | SEQ. ID. |
| | | NO. 107 | NO. 108 |

TABLE 5

| | bp | Fw | Rv |
|---|---|---|---|
| P57 | 791 | SEQ. ID. | SEQ. ID. |
| | | NO. 109 | NO. 110 |
| P58 | 518 | SEQ. ID. | SEQ. ID. |
| | | NO. 111 | NO. 112 |
| P59 | 675 | SEQ. ID. | SEQ. ID. |
| | | NO. 113 | NO. 114 |
| P60 | 587 | SEQ. ID. | SEQ. ID. |
| | | NO. 115 | NO. 116 |
| P61 | 600 | SEQ. ID. | SEQ. ID. |
| | | NO. 117 | NO. 118 |
| P62 | 756 | SEQ. ID. | SEQ. ID. |
| | | NO. 119 | NO. 120 |
| P63 | 512 | SEQ. ID. | SEQ. ID. |
| | | NO. 121 | NO. 122 |
| P64 | 472 | SEQ. ID. | SEQ. ID. |
| | | NO. 123 | NO. 124 |
| P65 | 518 | SEQ. ID. | SEQ. ID. |
| | | NO. 125 | NO. 126 |
| P66 | 556 | SEQ. ID. | SEQ. ID. |
| | | NO. 127 | NO. 128 |
| P67 | 529 | SEQ. ID. | SEQ. ID. |
| | | NO. 129 | NO. 130 |
| P68 | 182 | SEQ. ID. | SEQ. ID. |
| | | NO. 131 | NO. 132 |
| P69 | 294 | SEQ. ID. | SEQ. ID. |
| | | NO. 133 | NO. 134 |
| P70 | 342 | SEQ. ID. | SEQ. ID. |
| | | NO. 135 | NO. 136 |
| P71 | 550 | SEQ. ID. | SEQ. ID. |
| | | NO. 137 | NO. 138 |
| P72 | 699 | SEQ. ID. | SEQ. ID. |
| | | NO. 139 | NO. 140 |
| P73 | 655 | SEQ. ID. | SEQ. ID. |
| | | NO. 141 | NO. 142 |
| P74 | 317 | SEQ. ID. | SEQ. ID. |
| | | NO. 143 | NO. 144 |
| P75 | 566 | SEQ. ID. | SEQ. ID. |
| | | NO. 145 | NO. 146 |
| P76 | 401 | SEQ. ID. | SEQ. ID. |
| | | NO. 147 | NO. 148 |
| P77 | 496 | SEQ. ID. | SEQ. ID. |
| | | NO. 149 | NO. 150 |
| P78 | 395 | SEQ. ID. | SEQ. ID. |
| | | NO. 151 | NO. 152 |
| P79 | 582 | SEQ. ID. | SEQ. ID. |
| | | NO. 153 | NO. 154 |
| P80 | 363 | SEQ. ID. | SEQ. ID. |
| | | NO. 155 | NO. 156 |
| P81 | 534 | SEQ. ID. | SEQ. ID. |
| | | NO. 157 | NO. 158 |
| P82 | 593 | SEQ. ID. | SEQ. ID. |
| | | NO. 159 | NO. 160 |
| P83 | 780 | SEQ. ID. | SEQ. ID. |
| | | NO. 161 | NO. 162 |
| P84 | 502 | SEQ. ID. | SEQ. ID. |
| | | NO. 163 | NO. 164 |
| P85 | 661 | SEQ. ID. | SEQ. ID. |
| | | NO. 165 | NO. 166 |
| P86 | 457 | SEQ. ID. | SEQ. ID. |
| | | NO. 167 | NO. 168 |
| P87 | 660 | SEQ. ID. | SEQ. ID. |
| | | NO. 169 | NO. 170 |
| P88 | 422 | SEQ. ID. | SEQ. ID. |
| | | NO. 171 | NO. 172 |
| P89 | 676 | SEQ. ID. | SEQ. ID. |
| | | NO. 173 | NO. 174 |
| P90 | 415 | SEQ. ID. | SEQ. ID. |
| | | NO. 175 | NO. 176 |
| P91 | 267 | SEQ. ID. | SEQ. ID. |
| | | NO. 177 | NO. 178 |
| P92 | 703 | SEQ. ID. | SEQ. ID. |
| | | NO. 179 | NO. 180 |
| P93 | 567 | SEQ. ID. | SEQ. ID. |
| | | NO. 181 | NO. 182 |
| P94 | 546 | SEQ. ID. | SEQ. ID. |
| | | NO. 183 | NO. 184 |
| P95 | 484 | SEQ. ID. | SEQ. ID. |
| | | NO. 185 | NO. 186 |
| P96 | 559 | SEQ. ID. | SEQ. ID. |
| | | NO. 187 | NO. 188 |
| P97 | 480 | SEQ. ID. | SEQ. ID. |
| | | NO. 189 | NO. 190 |
| P98 | 798 | SEQ. ID. | SEQ. ID. |
| | | NO. 191 | NO. 192 |

One primer set was added to a single reaction solution, and shuttle PCR was carried out under the same reaction conditions for all primer sets, and these primer sets were confirmed to actually be able to amplify the target nucleic acids.

More specifically, 5 ng of Genome Mix and each of the primer sets were respectively added to 10 μL of 2× QIAGEN Multiplex PCR Master Mix (Qiagen) to final concentrations of the forward primers and reverse primers of each primer set of 0.1 μM to prepare 20 μL of each reaction solution. After treating the reaction solutions for 15 seconds at 95° C., PCR was carried out by carrying out 40 heat cycles, each consisting of 30 seconds at 94° C. and 6 minutes at 68° C., followed finally by carrying out an elongation reaction for 10 minutes at 68° C. Subsequently, 1 μl aliquots of the resulting 96 types of reaction solutions were recovered followed by separation and detection of the amplified target nucleic acids by electrophoresis.

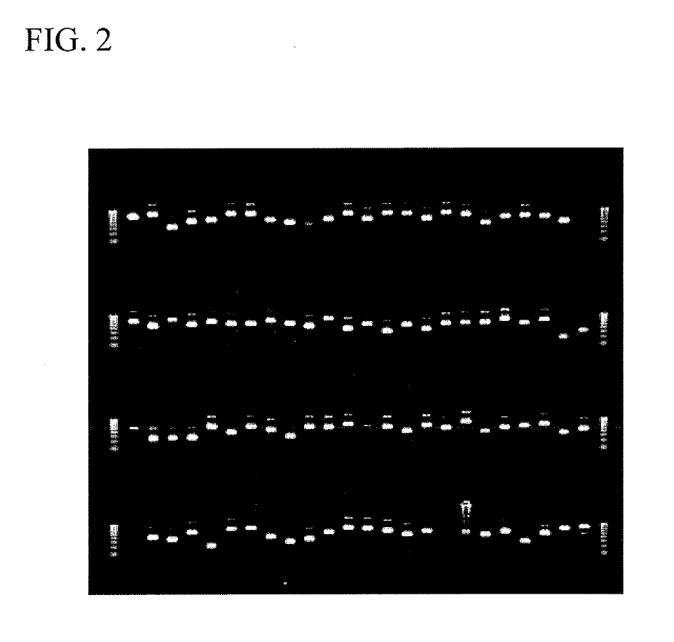

FIG. 2 shows the band patterns of the reaction solutions following PCR amplification of the 96 types of primer sets obtained by staining with ethidium bromide following agarose gel electrophoresis. The lanes on both ends of all four rows indicate the results of electrophoresing base pair length markers. Among the 96 types of primer sets of P3 to P98, the target nucleic acids were able to be confirmed to have been amplified in the reaction solutions of all 92 types of primer sets, with the exception of primer sets P39, P62, P89 and P95.

In other words, on the basis of the results of Example 2, primer sets for which reaction conditions had been standardized were clearly demonstrated to be able to amplify each target nucleic acid under the same reaction conditions.

### Example 3

Among the 92 types of primer sets confirmed to have been amplified in Example 2, 15 types of primer sets having different base pair lengths for the amplified target nucleic acids, consisting of primer sets P41, P31, P28, P26, P13, P88, P21, P44, P32, P30, P17, P87, P42, P52 and P14, were selected followed by carrying out amplification using 24 combinations thereof (sets A to X). Tables 6 to 8 show the base pair lengths of the target nucleotide sequences of each primer set along with the combinations of sets A to X. In the tables, “bp” indicates the base pair length of the target nucleic acid targeted for PCR amplification, while “O” indicates the primer sets contained in each set.

TABLE 6

| Set | bp | A | B | C | D | E | F | G | H |
|---|---|---|---|---|---|---|---|---|---|
| P14 | 792 | | | | | | | | |
| P52 | 742 | | | | | ◯ | ◯ | ◯ | ◯ |
| P42 | 703 | | | | | | | | |
| P87 | 660 | | | | | | | | |
| P17 | 632 | | | | | | | | |
| P30 | 579 | | | | ◯ | | ◯ | ◯ | |
| P32 | 530 | | | | | | | | |
| P44 | 508 | | | | | | | | |
| P21 | 462 | | | ◯ | | | ◯ | | |
| P88 | 422 | | | | | | | | |
| P13 | 382 | | | | | | | | |
| P26 | 358 | | | | | | | | |
| P31 | 277 | | ◯ | | | | | | ◯ |
| P28 | 249 | | | | | | | | |
| P41 | 181 | ◯ | | | | | | ◯ | ◯ |

TABLE 7

| Set | bp | I | J | K | L | M | N | O | P |
|---|---|---|---|---|---|---|---|---|---|
| P14 | 792 | | | | | | | | |
| P52 | 742 | | ◯ | ◯ | ◯ | ◯ | | ◯ | ◯ |
| P42 | 703 | | | | | | | | |
| P87 | 660 | | | | | | | | |
| P17 | 632 | | | | | | | | |
| P30 | 579 | | ◯ | ◯ | ◯ | | ◯ | ◯ | ◯ |
| P32 | 530 | | | | | | | | |
| P44 | 508 | | | | | | | | |
| P21 | 462 | ◯ | ◯ | ◯ | | ◯ | ◯ | ◯ | ◯ |
| P88 | 422 | | | | | | | | |
| P13 | 382 | | | | | | | | |
| P26 | 358 | | | | | | | | |
| P31 | 277 | ◯ | ◯ | | ◯ | ◯ | ◯ | ◯ | ◯ |
| P28 | 249 | | | | | | | | ◯ |
| P41 | 181 | ◯ | | ◯ | ◯ | ◯ | ◯ | ◯ | ◯ |

TABLE 8

| Set | bp | Q | R | S | T | U | V | W | X |
|---|---|---|---|---|---|---|---|---|---|
| P14 | 792 | | | | | | | | ◯ |
| P52 | 742 | ◯ | ◯ | ◯ | ◯ | ◯ | ◯ | ◯ | ◯ |
| P42 | 703 | | | | | | | ◯ | ◯ |
| P87 | 660 | | | | | | ◯ | ◯ | ◯ |
| P17 | 632 | | | | | ◯ | ◯ | ◯ | ◯ |
| P30 | 579 | ◯ | ◯ | ◯ | ◯ | ◯ | ◯ | ◯ | ◯ |
| P32 | 530 | | | | | ◯ | ◯ | ◯ | ◯ |
| P44 | 508 | | | | ◯ | ◯ | ◯ | ◯ | ◯ |
| P21 | 462 | ◯ | ◯ | ◯ | ◯ | ◯ | ◯ | ◯ | ◯ |
| P88 | 422 | | | ◯ | ◯ | ◯ | ◯ | ◯ | ◯ |
| P13 | 382 | | ◯ | ◯ | ◯ | ◯ | ◯ | ◯ | ◯ |
| P26 | 358 | ◯ | ◯ | ◯ | ◯ | ◯ | ◯ | ◯ | ◯ |
| P31 | 277 | ◯ | ◯ | ◯ | ◯ | ◯ | ◯ | ◯ | ◯ |
| P28 | 249 | ◯ | ◯ | ◯ | ◯ | ◯ | ◯ | ◯ | ◯ |
| P41 | 181 | ◯ | ◯ | ◯ | ◯ | ◯ | ◯ | ◯ | ◯ |

One set of sets A to X were added to a single reaction solution and shuttle PCR was carried out under the same reaction conditions. More specifically, 5 ng of Genome Mix and each of the primer sets were respectively added to 5 μL of 2× QIAGEN Multiplex PCR Master Mix (Qiagen) to final concentrations of 0.1 μM to prepare 10 μL of each reaction solution. PCR was carried out on the reaction solutions under the same reaction conditions as Example 2, and 1 μL aliquots of the resulting 24 types of reaction solutions were recovered followed by separation and detection of the amplified target nucleic acids by electrophoresis.

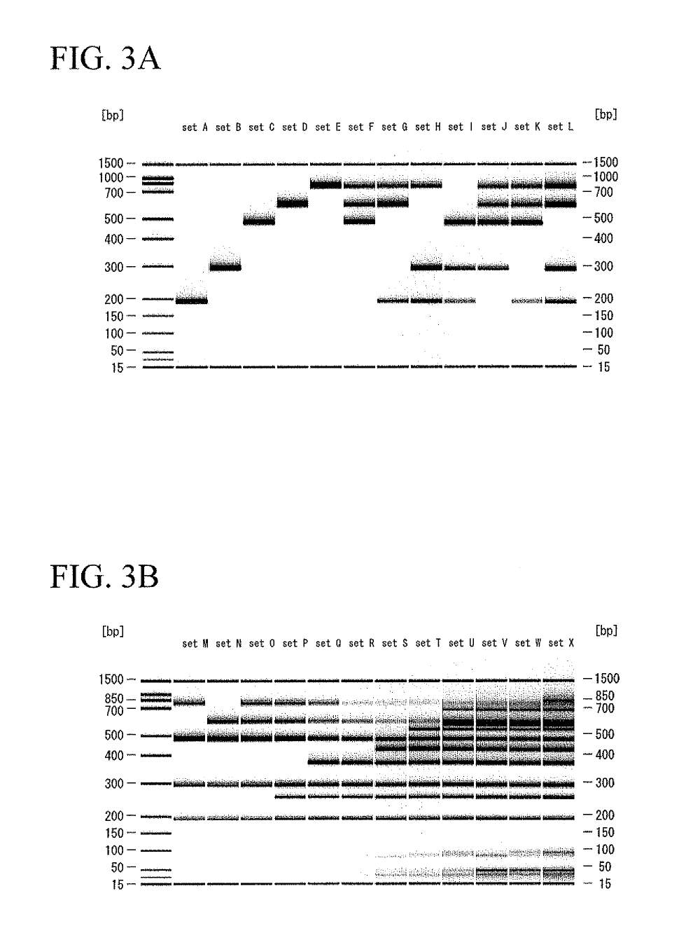

FIG. 3 shows the band patterns of the reaction solutions following PCR amplification as confirmed with a Model 2100 Bioanalyzer. FIG. 3(A) shows the band patterns of the reaction solutions to which sets A to L were added, while FIG. 3(B) shows the band patterns of the reaction solutions to which sets M to X were added. In the figures, the lane on the left end of the band patterns indicates the results of electrophoresing a base pair length marker. In addition, the bands for 1500 bp and 15 bp present in all lanes indicate the patterns for the phoretic dye. On the basis of these results, a primer kit composed of primers for which reaction conditions have been standardized was clearly demonstrated to be able to satisfactorily amplify each target nucleic acid by carrying out PCR on various combinations of primers without changing the reaction conditions.

The primer kit of the present invention can be used in fields such as gene analysis at health care institutions and the like for amplifying target nucleic acids using a wide range of specimens available in only small amounts by being able to efficiently amplify only a required target nucleic acid without shortage or excess.

What is claimed is:

1. A primer kit comprising:
a container; and
a plurality of types of primers for amplifying nucleic acids having a plurality of types of target nucleotide sequences;
wherein, all of the types of primers are primers capable of amplifying each nucleic acid having a target nucleotide sequence under the same reaction conditions, and a single type of primer or primers corresponding to a single target nucleotide sequence is/are housed in at least one said container.

2. A primer kit according to claim 1, wherein primers corresponding to a single target nucleotide sequence are housed in a single container.

3. A primer kit according to claim 1 wherein a single type of primer is housed in a single container.

4. A primer kit comprising:
a container; and
a plurality of types of primers for amplifying nucleic acids having a plurality of types of target nucleotide sequences;
wherein, all of the types of primers are primers capable of amplifying each nucleic acid having a target nucleotide sequence under the same reaction conditions, and primers corresponding to a single target nucleotide sequence are housed in the same container.

5. A primer kit according to any of claims 1 or 4, wherein the amplification is carried out by polymerase chain reaction (PCR).

6. A primer kit according to any of claims 1 or 4, wherein the base length of the primers is 30 bases or more.

7. A primer kit according to claim 5, wherein the reaction conditions are such that the sum of annealing time and elongation reaction time is 3 minutes or more.

8. A primer kit according to claim 6, wherein the reaction conditions are such that the sum of annealing time and elongation reaction time is 3 minutes or more.

9. A primer kit according to claim 5, wherein the reaction conditions are such that denaturation temperature, annealing temperature and elongation reaction temperature are all 68° C. or higher.

10. A primer kit according to claim 6, wherein the reaction conditions are such that denaturation temperature, annealing temperature and elongation reaction temperature are all 68° C. or higher.

11. A primer kit according to claim 9 or 10, wherein the reaction conditions are such that the annealing temperature and the elongation reaction temperature are the same.

12. A primer kit according to claim 5 containing heat-resistant DNA polymerase.

13. A primer kit according to claim 1 or 4, further comprising a reaction buffer.

14. A primer kit according to claim 1 or 4, further comprising a table containing target nucleotide sequences and combinations of primers used to amplify nucleic acids having those target nucleotide sequences.
